(19) United States
(12) Patent Application Publication
(10) Pub. No.: US 2010/0226888 A1
(43) Pub. Date: Sep. 9, 2010

(54) Ethanol Enhances the Complete Replication of Hepatitis C Virus: the Role of Acetaldehyde

(75) Inventors: Jinah Choi, Merced, CA (US); Scott Seronello, Merced, CA (US)

Correspondence Address: FOLEY & LARDNER LLP, 975 PAGE MILL ROAD, PALO ALTO, CA, 94304, US

(73) Assignee: The Regents of the University of California

(21) Appl. No.: 12/718,915

(22) Filed: Mar. 5, 2010

Related U.S. Application Data
(60) Provisional application No. 61/158,314, filed on Mar. 6, 2009.
(60) Provisional application No. 61/173,920, filed on Apr. 29, 2009.

(51) Int. Cl. A61K 38/21; C12Q 1/70; C12N 5/02; A61P 31/12

(52) U.S. Cl. 424/85.7; 424/85.4; 435/5; 435/375; 514/567

(57) ABSTRACT

Methods are provided for inhibiting replication of an RNA virus in a cell infected with the virus, wherein the cell is characterized as having been or concurrently being exposed to a physiologically relevant concentration of a compound selected from ethanol, acetate, isopropanol, acetaldehyde or acetone, comprising contacting the cell with an effective amount of one or more of a HMG-CoA reductase inhibitor, a fatty acid biosynthesis inhibitor, thyroxine, or an agent promoting clearance of the compound from a cell. Also provided are methods to treat a subject having one or more cells characterized as having a physiological concentration of ethanol, acetate, isopropanol, acetaldehyde or acetone, in particular subjects that suffer chronic alcoholics, diabetes or starvation.

### CROSS-REFERENCE TO RELATED APPLICATIONS

This application claims the benefit under 35 U.S.C. §119(d) of U.S. provisional application Ser. Nos. 61/173,920 and 61/158,314, filed Apr. 29, 2009 and Mar. 6, 2009, respectively. The contents of these applications are hereby incorporated by reference into the present disclosure.

### BACKGROUND

Hepatitis C virus (HCV) is a small, enveloped, positive-sense stranded RNA virus of the Flaviviridae family that is transmitted through blood. HCV is estimated to have infected 170 million individuals worldwide. Approximately 60-85% of HCV infections result in chronic infection that can lead to serious health complications including cirrhosis, steatosis, and hepatocellular carcinoma (HCC). There is currently no vaccine for HCV but anti-HCV therapy, which consists of PEGylated interferon-a (IFN-a) and ribavirin, achieves sustained virological response (SVR) in about 50-60% of individuals undergoing treatment. Despite continued research, the mechanism by which HCV interacts with various host and environmental factors to induce pathogenesis remains unclear.

Ethanol consumption is a well-known risk factor for chronic liver diseases. Ethanol is also a key cofactor in the pathogenesis induced by HCV and decreases the efficacy of anti-HCV treatments (2-4). Likewise, HCV infection exacerbates liver damage caused by prolonged alcohol abuse (3). It has also been reported that patients with a history of alcohol abuse are more likely to contract HCV than the rest of the population (2). The mechanism of pathological interactions between ethanol and HCV is unclear. However, HCV infection is associated with severe alterations of the host redox status with increased generation of reactive oxygen and nitrogen species (ROS/RNS) and decreased antioxidant defense (5). Thus, combined oxidative/nitrosative stress as well as the generation of acetaldehyde during ethanol metabolism have been suggested to play an important role (5).

In addition, ethanol may exacerbate HCV-induced liver diseases by affecting the viral titer (3, 5-9). Hepatitis C patients who drink alcohol typically show a pattern of hepatic injury that is more characteristic of chronic viral hepatitis than alcohol-induced injury, suggesting that alcohol enhances the pathogenic effects of HCV rather than exerting its independent effects on liver (10). Several clinical studies have correlated increased serum and intrahepatic HCV titer with the amount of alcohol consumed (3, 5-8, 11). HCV titer is significantly higher in patients consuming greater than 10 g of alcohol per day (12). Habitual drinkers also show higher levels of HCV RNA than non-habitual drinkers (8). Abstinence or moderation of alcohol consumption could reduce the HCV titer in some patients (3, 12). Furthermore, in vitro studies suggest that ethanol increases HCV RNA level in Huh7 human hepatoma replicon cell lines that continuously support the HCV RNA replication without virus production (9, 13, 14). These studies suggest that ethanol enhances HCV replication both in the presence and absence of the complete viral replication cycle. HCV replicon systems and more recent virus-producing cell culture models have increased our understanding of HCV and provide us with tools for studying potential interactions between HCV and pathological cofactors, such as ethanol. A recent review describing HCV replication cycle is found in reference (15). Nevertheless, whether ethanol directly enhances HCV production in the context of the complete viral replication cycle has not been demonstrated. In addition, the mechanism by which ethanol modulates HCV RNA replication remains controversial as ROS and lipid peroxidation products, which can be generated during ethanol metabolism, have been found to suppress, rather than increase, HCV RNA replication in cells (16-21).

### SUMMARY OF THE INVENTION

This invention provides a method for inhibiting replication of an RNA virus in a cell infected with the virus, wherein the cell is characterized as having been or concurrently being exposed to a physiologically relevant concentration of a compound selected from ethanol, acetate, isopropanol, acetaldehyde or acetone, comprising contacting the cell with an effective amount of one or more of a HMG-CoA reductase inhibitor, a fatty acid biosynthesis inhibitor, thyroxine, or an agent promoting clearance of the compound from a cell, thereby inhibiting replication of the virus in the cell.

Also provided is a method for inhibiting replication of an RNA virus in a cell infected with the virus, wherein the cell is characterized as having been or concurrently being exposed to a physiologically relevant concentration of a compound selected from ethanol, acetate, isopropanol, acetaldehyde or acetone, comprising contacting the cell with an effective amount of an agent selected from atovastatin, cerivastatin, fluvastatin, lovastatin, mevastatin, pitavastatin, pravastatin, rosuvastatin, simastatin, 5-(Tetradecyloxy)-2-furoic acid (TOFA), cerulenin, thyroxine, bezafibrate, ciprofibrate, clofibrate, gemifibrozil or fenofibrate, thereby inhibiting replication of the virus in the cell.

For the purpose of these inventions, the RNA virus can be a positive sense RNA virus such as a positive sense single strand RNA virus, or a negative sense RNA virus. Non-limiting examples of positive sense single strand RNA virus include Yellow fever viruses, West Nile viruses, Dengue Fever viruses and hepatitis C viruses (HCV). In one aspect, the RNA virus is an HCV virus. In another aspect, when the virus is an HCV virus, the method further comprises contacting the cell with an anti-HCV agent, examples of which include, without limitation, one which produces a subtoxic concentration of hydrogen peroxide. Additional anti-HCV agents included without limitiation interferon, plerixafor, ribavirin, pegylated interferon-alpha-2a or pegylated interferon-alpha-2b.

Any cell which is subject to infection by an RNA virus and wherein the cell is characterized as having been or concurrently being exposed to a physiologically relevant concentration of a compound selected from ethanol, acetate, isopropanol, acetaldehyde or acetone is encompassed by this invention, including but not limited to, a mammalian cell such as a human cell. The contacting may be in vitro or in vivo. When in vivo, the method is useful to treat a subject, such as a human patient infected with an RNA virus, wherein one or more cells in the subject is characterized as having been or concurrently being exposed to a physiological relevant concentration of a compound selected from ethanol, acetate, isopropanol, acetaldehyde or acetone, by administering an effective amount of the agent or agents, or compositions containing these agents, to the subject. Subjects that have one or more cells characterized as having been or concurrently exposed to a physiological relevant concentration of a compound selected from the group consisting of ethanol, acetate, isopropanol, acetaldehyde and acetone, for example, can be a subject that suffers chronic alcoholism, diabetes or is under physiological starvation.

This invention also provides a method for identifying an agent suitable for inhibiting replication of an RNA virus in a cell infected with the virus, wherein the cell is characterized as having been or concurrently being exposed to a physiological relevant concentration of a compound selected from ethanol, acetate, isopropanol, acetaldehyde or acetone, comprising contacting a first sample of the cell with a candidate agent and separately contacting a second sample of the cell with an effective amount of one or more of an HMG-CoA reductase inhibitor mevalonate pathway inhibitor, statins, a fatty acid biosynthesis inhibitor TOFA (5-(Tetradecyloxy)-2-furoic acid), cerulenin, thyroxine to decrease NADH/NAD+ ratio or an agent promoting clearance of the compound from a cell, wherein a decreased replication of the RNA virus in the cell substantially equal to or greater than the decreased replication of the RNA virsus in the second sample of the cell indicates that the candidate agent is suitable for inhibiting replication of the RNA virus in the cell. In a further aspect when the RNA virus is an HCV virus, the first and second samples can be contacted with an effective amount of an anti-HCV agent.

### DESCRIPTION OF THE FIGURES

FIG. 1. Ethanol increases JFH1 replication. Huh7 cells transfected with JFH1 RNA were analyzed for intracellular (A) and extracellular (B) HCV RNA by qRT-PCR or Northern blots after 48 hrs of ethanol treatments. Cell culture dishes were wrapped with parafilm during ethanol exposure. (C) Naive Huh7 cells were inoculated with virus-containing medium and analyzed for HCV RNA after 48 hrs of ethanol treatments. Indicates statistically significant difference (P<0.05).

FIG. 2. Ethanol increases the replication of subgenomic JFH1 and Con1 replicon RNAs. (A) Huh7 cells transfected with SgJFH1-Luc RNA were assayed for luciferase activity after 48 hr of ethanol treatments (n=3). (B) Stable Huh7 clones expressing SgConI-Neo (SgPC2) were incubated with ethanol for 24 hrs and analyzed for HCV RNA, GAPDH mRNA, and NS5A and β-actin proteins (n=3) by Northern and Western blots, respectively (n=3). (C-D) Cytostolic lysates were prepared from (C) JFH1 and JFH1-GND RNA-transfected cells and SgPC2 cells (D) were treated with ethanol for 5 hrs, and the cytosolic lysates were used for in vitro HCV RNA replication assays (n=3). Bottom panels show ethidium bromide staining of rRNA as the loading control. * Indicates statistically significant difference (P<0.05).

FIG. 3. CYP2E1 expression in Huh7 cells. (A) CYP2E1-dependent ethanol metabolism. (B) human liver tissue, Huh7 cells transfected with 50 μM non-targeting control or CYP2E1 siRNA, and skeletal muscle tissue were analyzed for CYP2E1 protein content by Western blot (n=3). (C and D) mock- or JFH1-transfected Huh7 cells were incubated with or without 0.2% (v/v) ethanol for 48 h and analyzed for (C) CYP2E1 expression by Western blot (n=3) and (D) CYP2E1-dependent p-nitrophenol hydroxylation activity (n=3). (E) SgPC2 cells were exposed to 0.2% ethanol±25 μM DADS for 24 h or transfected with 50 nM control or CYP2E1 siRNA for 24 h and then incubated with ethanol for 24 h and analyzed for HCVRNAby Northern blot (n=3). *, indicates statistically significant difference for indicated sample sizes (p<0.05).

FIG. 4. Endogenous and exogenous ROS suppress HCV replication. JFH1-transfected Huh7 cells were treated with BSO with and without 2 mM GSH or GSH ester (A, B), GO+glucose with and without 16 hr pre-treatment with 20 μM BSO (C), or bolus H₂O₂ (D) for 24 hrs. FIG. 4D shows that 25, 50, and 100 μM H₂O₂ can decrease the JFH1 RNA level in the cells tested. During the course of this study, Applicants found that while 25 and 50 μM H₂O₂ were clearly within the subtoxic range, the highest concentration of H₂O₂ used (100 μM) showed some cytotoxicity in the JFH1 cells. But even at this concentration, JFH1 RNA was significantly decreased compared to the control (0 μM H₂O₂, P<0.05, FIG. 4D), and there was no significant difference in the level of JFH1 RNA at 100 μM H₂O₂ versus 25 and 50 μM H₂O₂ (P<0.05, FIG. 4D). The 100 μM data point was removed in Applicants' publication (Seronelo et al. (2010), infra) to stay clearly within the subtoxic range as cell toxicity might introduce other variables that, too, would affect these cells. Then, JFH1 intracellular (A, C, D) and extracellular (B) HCV RNA levels were analyzed by qRT-PCR. (E) Huh7 cells transfected with SgJFH1-Luc RNA were assayed for luciferase activity after 24 hr treatment with 0.25 mU/mL glucose oxidase+glucose with and without the BSO pretreatment. * Indicates statistically significant difference (P<0.05).

FIG. 5. Acetaldehyde increases intracellular HCV RNA. SgJFH1-Luc (A), JFH1 RNA-transfected cells (B), Huh7.5 cells inoculated with JFH1 virus-containing medium (C), SgPC2 (D), and CloneB cells (E) were incubated with acetaldehyde for 24 hrs and analyzed for HCV RNA by Northern blot or qRT-PCR. (n=3)* Indicates statistically significant difference for indicated sample size (P<0.05).

FIG. 6. Role of NADH/NAD+ in the potentiation of HCV replication by ethanol, acetaldehyde, acetate, isopropyl alcohol, and acetone. SgPC2 cells, supporting Con1 subgenomic HCV RNA replication, were treated with (A) 0.2% ethanol±0.1 mM 4 MP plus 25 μM DADS or 0.1 mM cyanamide (n=3); (B) 0.2% ethanol, 5 μM acetaldehyde, 5 μM acetate, 0.2% isopropyl alcohol, 2 mM acetone, or 25 mM tert-butanol (n=4); (C) 0.2% ethanol, 5 μM acetaldehyde, 5 μM acetate, 0.2% isopropyl alcohol, and 2 mM acetone, with and without 5 mM pyruvate (n=3); or (D) 0.2% ethanol or 5 mM lactate for 3 h for NADH/NAD+ ratio measurement or 24 h for HCV RNA levels. HCV RNA levels were monitored by Northern blot (A-D, left panels). NADH/NAD+ ratios were measured by an enzymatic NADH recycling assay. Northern blots were quantified by densitometry. *, indicates statistically significant difference for indicated sample sizes (p<0.05).

FIG. 7. Role of lipogenesis in the enhancement of HCV replication by ethanol, acetaldehyde, isopropyl alcohol, acetone, and acetate. SgPC2 cells were treated for 24 h with (A and B) 0.2% ethanol, 5 μM acetaldehyde, 0.2% isopropyl alcohol, 2 mM acetone, 5 μM acetate±30 min pretreatment with (A) 5 μM lovastatin, 5 μM fluvastatin, (B) 5 μg/ml TOFA, 5 μg/ml cerulenin, or with (C) 2 mM β-mercaptopropionic acid (β-MPA). Then, HCV RNA levels were monitored by Northern blot and quantified by densitometry (n=3). D, SgPC2 cells, treated for 24 h with ethanol, acetaldehyde, acetone, and acetate±lovastatin, were monitored for cholesterol levels (n=3). Lovastatin was activated, as described, before use (29). *, indicates statistically significant difference for indicated sample sizes (p<0.05)

### DETAILED DESCRIPTION OF THE INVENTION

### Abbreviations

The use of the following abbreviations facilitate the description of the disclosed inventions: ALT, alanine aminotransferase; BCA, bicinchoninic acid; BSO, L-buthionine S,R-sulfoximine; DMEM Dulbecco's Modified Eagle Medium; DUI, driving under the influence; EMCV, encephalomyocarditis virus; ER, endoplasmic reticulum; FBS, fetal bovine serum; GAPDH, glyceraldehyde 3-phosphate dehydrogenase; GO, glucose oxidase; GSH, glutathione; HCV, hepatitis C virus; HCC, hepatocellular carcinoma; HMG-CoA reductase, 3-hydroxy-3-methyl-glutaryl-CoA reductase; IFN-a, interferon-a; IRES, internal ribosomal entry site; KRPH, Krebs-Ringer/Phosphate/Hepes; NAC, N-acetylcysteine; NADH/NAD+, nicotinamide adenine dinucleotide; NF-_{K}B, nuclear factor kappa B; nt., nucleotides; qRT-PCR, quantitative reverse transcriptase-polymerase chain reaction; RNS, reactive nitrogen species; ROS, reactive oxygen species; SVR, sustained virological response; TOFA, 5-(Tetradecyloxy)-2-furoic acid; UTR, untranslated region.

### DEFINITIONS

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

Throughout this application various technical publications are referenced directly or by reference to an Arabic numeral. Complete bibliographic citations for the Arabic-referenced citations can be found at the end of the specification, immediately preceding the claims. The disclosures of these publications are incorporated by reference into the present disclosure to more fully describe the state of the art to which this invention pertains.

The practice of the present invention will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Techique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)).

All numerical designations, e.g., pH, temperature, time, concentration, and molecular weight, including ranges, are approximations which are varied (+) or (−) by increments of 0.1 or 1.0, where appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a cell” includes a plurality of cells, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The term “isolated” as used herein with respect to nucleic acids, such as DNA or RNA, refers to molecules separated from other DNAs or RNAs, respectively that are present in the natural source of the macromolecule. The term “isolated nucleic acid” is meant to include nucleic acid fragments which are not naturally occurring as fragments and would not be found in the natural state. The term “isolated” is also used herein to refer to polypeptides, proteins and/or host cells that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

As is known to those of skill in the art, there are 6 classes of viruses. The DNA viruses constitute classes I and II. The RNA viruses and retroviruses make up the remaining classes. Class III viruses have a double-stranded RNA genome. Class IV viruses have a positive single-stranded RNA genome, the genome itself acting as mRNA Class V viruses have a negative single-stranded RNA genome used as a template for mRNA synthesis. Class VI viruses have a positive single-stranded RNA genome but with a DNA intermediate not only in replication but also in mRNA synthesis. Retroviruses carry their genetic information in the form of RNA; however, once the virus infects a cell, the RNA is reverse-transcribed into the DNA form which integrates into the genomic DNA of the infected cell. The integrated DNA form is called a provirus.

HCV is a member of the Flavivirus family. Others include, but are not limited to GB virus B, Japanese Encephalovirus (JEV) and West Nile Virus (WNV).

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

A polynucleotide or polynucleotide region (or a polypeptide or polypeptide region) has a certain percentage (for example, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by ═HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/cgi-bin/BLAST.

The expression “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

A “gene” refers to a polynucleotide containing at least one open reading frame (ORF) that is capable of encoding a particular polypeptide or protein after being transcribed and translated. Any of the polynucleotide or polypeptide sequences described herein may be used to identify larger fragments or full-length coding sequences of the gene with which they are associated. Methods of isolating larger fragment sequences are known to those of skill in the art.

The term “express” refers to the production of a gene product.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

A “gene product” or alternatively a “gene expression product” refers to the amino acid (e.g., peptide or polypeptide) generated when a gene is transcribed and translated.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “genotype” refers to the specific allelic composition of an entire cell or a certain gene, whereas the term “phenotype” refers to the detectable outward manifestations of a specific genotype. Viral genotype refers to specific genetic composition of a viral genome.

A “probe” when used in the context of polynucleotide manipulation refers to an oligonucleotide that is provided as a reagent to detect a target potentially present in a sample of interest by hybridizing with the target. Usually, a probe will comprise a detectable label or a means by which a label can be attached, either before or subsequent to the hybridization reaction. Alternatively, a “probe” can be a biological compound such as a polypeptide, antibody, or fragments thereof that is capable of binding to the target potentially present in a sample of interest.

“Detectable labels” include, but are not limited to radioisotopes, fluorochromes, chemiluminescent compounds, dyes, and proteins, including enzymes. Detectable labels can also be attached to a polynucleotide, polypeptide, antibody or composition described herein.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in MacPherson et al. (1991) PCR 1: A Practical Approach (IRL Press at Oxford University Press). All processes of producing replicate copies of a polynucleotide, such as PCR or gene cloning, are collectively referred to herein as “replication.” A primer can also be used as a probe in hybridization reactions, such as Southern or Northern blot analyses. Sambrook and Russell (2001), supra.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC. Hybridization reactions can also be performed under “physiological conditions” which is well known to one of skill in the art. A non-limiting example of a physiological condition is the temperature, ionic strength, pH and concentration of Mg²⁺ normally found in a cell.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

The term “propagate” means to grow a cell or population of cells. The term “growing” also refers to the proliferation of cells in the presence of supporting media, nutrients, growth factors, support cells, or any chemical or biological compound necessary for obtaining the desired number of cells or cell type.

The term “culturing” refers to the in vitro propagation of cells or organisms on or in media of various kinds. It is understood that the descendants of a cell grown in culture may not be completely identical (i.e., morphologically, genetically, or phenotypically) to the parent cell.

The term “inhibiting replication of an RNA virus” intends to inhibit or reduce the rate of any step of the RNA viral replication.

The term “physiological concentration” or “physiologically relevant concentration” as used herein refers to a concentration of a chemical or biological molecule that can be attained in biological systems in life settings, such as blood concentration of alcohol of a person drinking alcohol. Therefore, “physiological concentration” or “physiologically relevant concentration” here could refer to either toxic or subtoxic concentrations of chemicals or biological molecules as long as it is within the concentration range that can be found in biological systems (e.g., in human body).

“An agent that promotes clearance of the compound from a cell” or “an agent that promotes clearance of the volatile compound from a cell” intends a chemical or biological molecule that enhances a cell's capability to remove one or more of the volatile compounds as described herein from the cell. Non-limiting examples of such agents include bezafibrate, ciprofibrate, clofibrate, gemifibrozil, fenofibrate or equivalents thereof including other fibrate compounds.

The term “a subtoxic concentration of hydrogen peroxide” intends a concentration of hydrogen peroxide in a cell or tissue that is does not cause toxicity or does not cause a level of toxicity sufficient to substantially deteriorate the biological behavior or function of the cell or tissue.

As used herein the term “starvation” when used to describe a subject, intends a reduction in vitamin, nutrient, and energy intake for a period of time sufficient to cause malnutrition leading to increased level of ketone bodies.

“Hydrogen peroxide” is intended to mean H₂O₂, and is intended encompass a common precursor “superoxide” and other reactive oxygen species.

“Ascorbate” is intended to mean 2-oxo-L-threo-hexono-1,4-lactone-2,3-enediol, (R)-3,4-dihydroxy-5-((S)-1,2-dihydroxyethyl)furan-2(5H)-one, ascorbic acid or vitamin-C or the ionized form thereof. “Dehydroascorbate” is intended to mean dehydroascorbic acid (DHA) or the ionized form thereof, or an oxidized form of ascorbate.

“NAD(P)H oxidase” is intended to mean nicotinamide adenine dinucleotide phosphate-oxidase. NAD(P)H oxidase is or “NOx Protiens” Suitable compounds for inclusion in the methods of this invention include, for example, other sources of reactive oxygen species include the NADPH oxidases, xanthine oxidase, uncoupled nitric oxide synthase, and mitochondrial sources

“BCNU” is intended to mean 1,3-bis(chloroethyl)-1-nitrosourea. BCNU, also known as Carmustine, is used as an alkylating agent in chemotherapy. Carmustine for injection is marketed under the name BiCNU by Bristol-Myers Squibb.

“Quinone” is intended to mean a cyclohexadienedione compound or derivative thereof. Derivatives of such compounds include, but are not limited to tert-butylhydroquinone (TBHQ). These compounds are commercially available from sources such as Sigma. Suitable compounds for inclusion in the methods of this invention include, for example, 1,4-naphthoquinone, 5-hydroxy-1,4-naphthoquinone, 2-hydroxy-1,4-naphthoquinone, 2-methyl-1,4-naphthoquinone (menadione), 5-hydroxy-2-methyl-1,4-naphthoquinone, 3-hydroxy-2-methyl-1,4-naphthoquinone, 2,3-dimethyl-1,4-naphthoquinone and 2,3-dimethoxy-1,4-naphthoquinone (DMNQ). Suitable compounds for inclusion in the methods of this invention also include quinone anticancer agents, for example, diazyquone (AZQ), andriamycin, 2,5-diaziridinyl-1,4-benzoquinone (DZQ), and derivatives thereof.

“Butylated hydroxyanisole” or “BHA” is intended to mean a mixture of 2-tert-butyl-4-hydroxyanisole and 3-tert-butyl-4-hydroxyanisole. Suitable compounds for inclusion in the methods of this invention include, for example, other butylated phenols such as butylated hydroxytoluene (BHT). BHA and BHT can be purchased from Sigma.

A “composition” is intended to mean a combination of active polypeptide, polynucleotide or antibody and another compound or composition, inert (e.g. a detectable label or a pharmaceutically acceptable carrier) or active (e.g. a gene delivery vehicle).

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

A “subject,” “individual” or “patient” is used interchangeably herein, and refers to a vertebrate, preferably a mammal, more preferably a human. Mammals include, but are not limited to, murines, rats, rabbit, simians, bovines, ovine, porcine, canines, feline, farm animals, sport animals, pets, equine, and primate, particularly human. Besides being useful for human treatment, the present invention is also useful for veterinary treatment of companion mammals, exotic animals and domesticated animals, including mammals, rodents, and the like which are susceptible to RNA viral infection. In one embodiment, the mammals include horses, dogs, and cats. In another embodiment of the present invention, the human is an adolescent or infant under the age of eighteen years of age.

“Host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The terms “disease,” “disorder,” and “condition” are used inclusively and refer to any condition mediated at least in part by infection by an RNA virus such as HCV.

Treating” or “treatment” of a disease includes: (1) preventing the disease, i.e., causing the clinical symptoms of the disease not to develop in a patient that may be predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

The term “suffering” as it related to the term “treatment” refers to a patient or individual who has been diagnosed with or is predisposed to infection or a disease incident to infection. A patient may also be referred to being “at risk of suffering” from a disease because of active or latent infection. This patient has not yet developed characteristic disease pathology.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks. Consistent with this definition, as used herein, the term “therapeutically effective amount” is an amount sufficient to inhibit RNA virus replication in vitro or in vivo.

The term administration shall include without limitation, administration by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of administration, the formulation or dosing schedule.

### Methods to Inhibit RNA Virus Replication

It has now been discovered that volatile compounds such as ethanol, acetate, isopropanol, acetaldehyde and acetone, at subtoxic and physiologically relevant concentrations, enhance complete RNA viral replication. The potentiation of RNA viral infection by these volatile compounds can be suppressed by inhibiting CYP2E1 or inhibiting aldehyde dehydrogenase and requires elevated NADH/NAD+ ratio. It has also been demonstrated that by inhibiting the mevalonate pathway with statins such as lovastatin or fluvastatin, or by inhibiting the fatty acid synthesis with TOFA or cerulenin, the enhancement of RNA rival replication of the volatile compounds can be attenuated. Therefore, by contacting a cell that has or has been exposed to a subtoxic and physiologically relevant concentration of one or more these volatile compounds with an agent that inhibits one or more of the mevalonate pathway or inhibits the HMG-CoA reductase, inhibits fatty acid biosynthesis, decreases the NADH/NAD+ ratio, or promotes clearance of the volatile compound from the cell, one can inhibit the RNA viral replication in the cell.

Certain subjects with compromised liver function, such as individuals that suffer chronic alcoholicism, diabetes or starvation, contain cells, such as liver cells, characterized as having or having been exposed to a physiological relevant concentration of one or more of these volatile compounds. This invention, therefore, is helpful in preventing RNA viral infection or inhibiting RNA viral replications in these subjects.

Accordingly, this invention, in one aspect, provides a method for inhibiting replication of an RNA virus in a cell infected with the virus, wherein the cell is characterized as having been or concurrently being exposed to a physiologically relevant concentration of a compound selected from ethanol, acetate, isopropanol, acetaldehyde or acetone, comprising, or alternatively consisting essentially of, or yet further consisting of, contacting the cell with an effective amount of one or more of a HMG-CoA reductase inhibitor, a fatty acid biosynthesis inhibitor, thyroxine, or an agent promoting clearance of the compound from a cell, thereby inhibiting replication of the virus in the cell.

HMG-CoA reductase inhibitors are also inhibitors of the mevalonate pathway. Non-limiting examples include atovastatin, cerivastatin, fluvastatin, lovastatin, mevastatin, pitavastatin, pravastatin, rosuvastatin, simastatin or equivalents thereof including other statin compounds.

Fatty acid biosynthesis inhibitors can be 5-(Tetradecyloxy)-2-furoic acid (TOFA) or cerulenin.

Thyroxine, also known as 3,5,3′,5′-tetraiodothyronine and often abbreviated as T4, is a form of thyroid hormones that can decrease the NADH/NAD+ ratio.

Agents promoting clearance of the compound from a cell can be bezafibrate, ciprofibrate, clofibrate, gemifibrozil, fenofibrate or equivalents thereof including other fibrate compounds.

One can determine when the RNA viral replication has been inhibited by use of PCR techniques described herein or by noting an increase in survival of the cells in culture.

Also provided is a method for inhibiting replication of an RNA virus in a cell infected with the virus, wherein the cell is characterized as having been or concurrently being exposed to a physiologically relevant concentration of a compound selected from ethanol, acetate, isopropanol, acetaldehyde or acetone, comprising, or alternatively consisting essentially of, or yet further consisting of contacting the cell with an effective amount of an agent selected from atovastatin, cerivastatin, fluvastatin, lovastatin, mevastatin, pitavastatin, pravastatin, rosuvastatin, simastatin, 5-(Tetradecyloxy)-2-furoic acid (TOFA), cerulenin, thyroxine, bezafibrate, ciprofibrate, clofibrate, gemifibrozil or fenofibrate, thereby inhibiting replication of the virus in the cell.

As used herein, any suitable cell that supports RNA viral reproduction and genomic replication is suitable for this method. Examples of such include, eukaryotic cells such as animals, e.g., murines, rats, rabbit, simians, bovines, ovine, porcine, canines, feline, farm animals, sport animals, pets, equine, and primate, particularly human. The cells can be cultured cells or they can be primary cells. Cultured cell lines can be purchased from vendors such as the American Type Culture Collection (ATCC), U.S.A. In one particular embodiment, the cells are liver cells, e.g., cultured liver cells or primary liver cells. The cells are infected with RNA virus and may further exhibit pathology such as a liver carcinoma.

Suitable agents that produces a subtoxic concentration of hydrogen peroxide include, for example, an agent from the group enzymatic generation with glucose oxidase and glucose, L-buthionine S,R-sulfoximine (BSO) or other agent that decreases antioxidant defense of the cell and therefore amplifies the effects of the endogenously generated reactive oxygen species, tTert-butylhydroquinone (TBHQ)—a redox cycling quinine, 2,3 dimethoxy-1,4-naphthoquinone (DMNQ)—a redox cycling quinine, ascorbate, dehydroascorbate, agents that induce and/or activate NAD(P)H oxidase family proteins (Nox proteins), BCNU or other inhibitor of glutathione reductase that decreases antioxidant defense of the cell and therefore amplifies the effects of the endogenously generated oxidants, menadione or other redox cycling quinine, diazyquone (AZQ), adriamycin, 2,5-diaziridinyl-1,4-benzoquinone (DZQ) or other quinone anticancer agents or butylated hydroxyanisole (BHA) that produces TBHQ.

In one aspect, the agent is BSO. In another aspect, the agent is a combination of an effective amount of hydrogen peroxide and L-buthionine S,R-sufloximine (BSO). In a further aspect, the agent is hydrogen peroxide alone or in combination with BSO. In another aspect, the agent is a combination of glucose and glucose oxidase. The agents, alone or in combination, can be formulated into pharmaceutical compositions or they can be directly contacted with the cell.

Suitable examples of RNA viruses that are inhibited by the methods of this invention include, but are not limited to, Flavivirus, (e.g., HCV). Other viruses that may be affected similarly will include Dengue virus, yellow fever virus and West Nile Virus. Another virus known to be inhibited by hydrogen peroxide is hepatitis B virus, a DNA virus that also infects and damages liver.

In one aspect, when the virus is HCV, the method can further comprise, or alternatively consisting essentially of, or yet further consist of contacting the cell with an anti-HCV agent, which may be in some aspects, one which produces a subtoxic concentration of hydrogen peroxide. Alternatively, the method can further comprise the administration of an effective amount of interferon, plerixafor, ribavirin, pegylated interferon-alpha-2a or pegylated interferon-alpha-2b.

In one aspect of the above methods, the agent produces a subtoxic concentration of hydrogen peroxide is one that increases endogenous reactive oxygen species (ROS). Methods to determine endogenous ROS are known in the art. Methods to determine if RNA viral replication has been reduced or inhibited also are known in the art and briefly described herein.

The methods can also be practiced by contacting with an agent that produces mild endogenous oxidative stress. In an alternate embodiment, the agent reduces intracellular glutathione.

The methods can be practiced in vitro or in vivo. When practiced in vitro, they are effective means to identify and test therapeutic agents and regimens before advancement into the clinic. By having two cell systems, one can test or screen a potential therapeutic and compare its activity to those agents and combinations described herein. Therefore, a method is provided for identifying or screening an agent suitable for inhibiting replication of an RNA virus in a cell infected with the virus, wherein the cell is characterized as having been or is concurrently exposed to a physiological relevant concentration of a compound selected from ethanol, acetate, isopropanol, acetaldehyde or acetone, comprising, or alternatively consisting essentially of, or yet further consisting of, contacting a first sample of the cell with a candidate agent and a second sample of the cell with an agent noted herein effective for the purpose of inhibiting viral replication in the cell and assaying for reduction of viral replication in the first and second cell sample. Methods to determine whether viral replication has been inhibited are known in the art and described herein. When the test agent in the first sample inhibits viral replication in an amount that is substantially equivalent to or greater than that observed in the second sample, then the candidate agent is suitable for inhibiting replication of the RNA virus in the cell. The methods can be modified for high-throughput testing of agents and potential therapeutics.

In vivo practice of the invention in an animal such as a rat or mouse provides a convenient animal model system that can be used prior to clinical testing of the agent. In this system, a potential agent, compound or composition will be successful if retroviral replication is reduced or the symptoms of the infection are ameliorated as compared to an untreated, infected animal. It also can be useful to have a separate negative control group of cells or animals which has not been infected, which provides a basis for comparison or alternatively, treated with an agent noted herein to be effective for this purpose of inhibiting RNA viral replication. In one aspect, the animal is under certain stress, such as reduced food intake to induce a starvation response, or has liver damage such as that suffered from alcoholism or diabetes.

When practiced in vivo, the candidate is administered or delivered to the animal in effective amounts. As used herein, the term “administering” for in vivo and ex vivo purposes means providing the subject with an effective amount of the candidate agent effective to inhibit retroviral replication as described herein. In these instances, the agent, compound or composition may be administered with a pharmaceutically acceptable carrier. These agents and combinations also can be used in the manufacture of medicaments and for the treatment of humans and other animals by administration in accordance with conventional procedures, such as an active ingredient in pharmaceutical compositions.

This invention provides a method for treating a subject infected with an RNA virus, wherein one or more cells in the subject is characterized as having been or concurrently being exposed to a physiologically relevant concentration of a compound selected from ethanol, acetate, isopropanol, acetaldehyde or acetone, comprising, or alternatively consisting essentially of, or yet further consisting of, administering to the subject an effective amount of one or more of a HMG-CoA reductase inhibitor, a fatty acid biosynthesis inhibitor, thyroxine or an agent promoting clearance of the compound from a cell, thereby treating the subject by inhibiting replication of the virus in the cell.

HMG-CoA reductase inhibitors are also inhibitors of the mevalonate pathway. Non-limiting examples include atovastatin, cerivastatin, fluvastatin, lovastatin, mevastatin, pitavastatin, pravastatin, rosuvastatin, simastatin or equivalents thereof including other statin compounds.

Fatty acid biosynthesis inhibitors can be 5-(Tetradecyloxy)-2-furoic acid (TOFA) or cerulenin.

Agents promoting clearance of the compound from a cell can be bezafibrate, ciprofibrate, clofibrate, gemifibrozil, fenofibrate or equivalents thereof including other fibrate compounds.

The methods are useful to inhibit the replication of an RNA virus. Suitable examples of RNA viruses which infect humans include, but are not limited to Flavivirus, e.g., HCV. Other viruses that may be affected similarly will include Dengue virus, yellow fever virus and West Nile Virus. Another virus known to be inhibited by hydrogen peroxide is hepatitis B virus, a DNA virus that also infects and damages the liver.

In one aspect, the method further comprises contacting the cell with an anti-HCV agent, which may be in some aspects, one which produces a subtoxic concentration of hydrogen peroxide. Additional anti-HCV agents include for example interferon, plerixafor, ribavirin, pegylated interferon-alpha-2a or pegylated interferon-alpha-2b.

Suitable examples of RNA viruses that are inhibited by these methods include, but are not limited to, Flavivirus, (e.g., HCV). Other viruses that may be affected similarly will include Dengue virus, yellow fever virus and West Nile Virus. Another virus known to be inhibited by hydrogen peroxide is hepatitis B virus, a DNA virus that also infects and damages liver.

Also provided is a method for treating a subject infected with or preventing a subject from infection by a RNA virus, wherein one or more cells in the subject are characterized as having been or concurrently exposed to a physiological relevant concentration of a compound selected from the group consisting of ethanol, acetate, isopropanol, acetaldehyde and acetone, comprising, or alternatively consisting essentially of, or yet further consisting of, administering to the subject an effective amount of an agent selected from atovastatin, cerivastatin, fluvastatin, lovastatin, mevastatin, pitavastatin, pravastatin, rosuvastatin, simastatin, TOFA, cerulenin, thyroxine, bezafibrate, ciprofibrate, clofibrate, gemifibrozil or fenofibrate.

A subject in need thereof may be animals, e.g., murines, rats, rabbit, simians, bovines, ovine, porcine, canines, feline, farm animals, sport animals, pets, equine, and primate, particularly human.

The subject that is characterized as having a cell containing a physiological relevant concentration of a compound selected from ethanol, acetate, isopropanol, acetaldehyde or acetone can be a subject that suffers chronic alcoholicism, diabetes or starvation.

Suitable agents that produces a subtoxic concentration of hydrogen peroxide for use in this method include, for example, an agent from the group enzymatic generation with glucose oxidase and glucose, L-buthionine S,R-sulfoximine (BSO) or other agent that decreases antioxidant defense of the cell and therefore amplifies the effects of the endogenously generated reactive oxygen species, tTert-butylhydroquinone (TBHQ)—a redox cycling quinine, 2,3 dimethoxy-1,4-naphthoquinone (DMNQ)—a redox cycling quinine, ascorbate, dehydroascorbate, agents that induce and/or activate NAD(P)H oxidase family proteins (Nox proteins), BCNU or other inhibitor of glutathione reductase that decreases antioxidant defense of the cell and therefore amplifies the effects of the endogenously generated oxidants, menadione or other redox cycling quinine, diazyquone (AZQ), adriamycin, 2,5-diaziridinyl-1,4-benzoquinone (DZQ) or other quinone anticancer agents or butylated hydroxyanisole (BHA) that produces TBHQ.

In one aspect, the agent is BSO. In another aspect, the agent is a combination of an effective amount of hydrogen peroxide and L-buthionine S,R-sufloximine (BSO). In a further aspect, the agent is hydrogen peroxide alone or in combination with BSO. In another aspect, the agent is a combination of glucose and glucose oxidase. The agents, alone or in combination, can be formulated into pharmaceutical compositions or they can be directly contacted with the cell.

The methods of this invention present unexpected advantage by inhibiting or reducing subgenomic viral replication without virus production. Methods to determine subgenomic viral replication are known in the art and briefly described herein. An additional unexpected advantage is that the methods inhibit the complete retroviral replication cycle. Methods to determine if the complete retroviral life cycle has been completed are known in the art and briefly described herein.

Also provided herein is a method for treating diseases incident to RNA viral infection, e.g., liver disease incident to Hepatitis C Viral infection, in a subject by use of a method of this invention as described above or alternatively by administering to the subject an effective amount of an agent that produces a subtoxic concentration of hydrogen peroxide in the subject. The agents can generate hydrogen peroxide or enhance endogenous levels of hydrogen peroxide. The agents are effective against HCV independent of genotype.

A subject in need thereof may be animals, e.g., murines, rats, rabbit, simians, bovines, ovine, porcine, canines, feline, farm animals, sport animals, pets, equine, and primate, particularly human.

Examples of liver disease incident to HCV include, but are not limited to cirrhosis, steatosis or hepatocellular carcinoma.

Subjects that are suitably treated are described above and include any animal, vertebrate or mammal that is susceptible to RNA viral, and for example, HCV infection. Persons at risk for HCV infection include injecting drug users, recipients of clotting factors made before 1987, hemodialysis patients, recipients of blood and/or solid organs before 1992, people with undiagnosed liver problems, infants born to infected mothers and healthcare/public safety workers after known exposure.

### Compositions

This invention also provides compositions containing the active agent as described herein to inhibit RNA viral replication. A “composition” typically intends a combination of the active agent and another carrier, e.g., compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this invention, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The term carrier further includes a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Additional carriers include polymeric excipients/additives such as polyvinylpyrrolidones, ficolls (a polymeric sugar), dextrates (e.g., cyclodextrins, such as 2-hydroxypropyl-.quadrature.-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives and any of the above noted carriers with the additional provisio that they be acceptable for use in vivo. For examples of carriers, stabilizers and adjuvants, see Martin REMINGTON'S PHARM. SCI., 15th Ed. (Mack Publ. Co., Easton (1975) and Williams & Williams, (1995), and in the “PHYSICIAN'S DESK REFERENCE”, 52^{nd} ed., Medical Economics, Montvale, N.J. (1998).

An “effective amount” of the agent or composition is contacted with the cell, in vitro or can be administered to the subject such as a human patient, in vivo. An effective amount is an amount sufficient to effect beneficial or desired results. An effective amount can be administered in one or more administrations, applications or dosages.

The invention provides an article of manufacture, comprising packaging material and at least one vial comprising a solution of at least one agent or composition with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further comprises an article of manufacture, comprising packaging material, a first vial comprising at least one agent or composition and a second vial comprising an aqueous diluent of prescribed buffer or preservative, wherein said packaging material comprises a label that instructs a patient to reconstitute the therapeutic in the aqueous diluent to form a solution that can be held over a period of twenty-four hours or greater.

In some aspects, the agent or composition is prepared to a concentration includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

The formulations of the present invention can be prepared by a process which comprises mixing at least one agent or composition and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing of the antibody and preservative in an aqueous diluent is carried out using conventional dissolution and mixing procedures. For example, a measured amount of at least one antibody in buffered solution is combined with the desired preservative in a buffered solution in quantities sufficient to provide the antibody and preservative at the desired concentrations. Variations of this process would be recognized by one of skill in the art, e.g., the order the components are added, whether additional additives are used, the temperature and pH at which the formulation is prepared, are all factors that can be optimized for the concentration and means of administration used.

The compositions and formulations can be provided to patients as clear solutions or as dual vials comprising a vial of agent or composition that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available. Recognized devices comprising these single vial systems include pen-injector devices for delivery of a solution such as BD Pens, BD Autojectore, Humaject®, NovoPen®, B-D®Pen, AutoPen®, and OptiPen®, GenotropinPen®, Genotronorm Pen®, Humatro Pen®, Reco-Pen®, Roferon Pen®, Biojector®, Iject®, J-tip Needle-Free Injector®, Intraject®, Medi-JectO, e.g., as made or developed by Becton Dickensen (Franklin Lakes, N.J. available at bectondickenson.com), Disetronic (Burgdorf, Switzerland, available at disetronic.com; Bioject, Portland, Oreg. (available at bioject.com); National Medical Products, Weston Medical (Peterborough, UK, available at weston-medical.com), Medi-Ject Corp (Minneapolis, Minn., available at mediject.com).

Various delivery systems are known and can be used to administer a therapeutic agent of the invention, e.g., encapsulation in liposomes, microparticles, microcapsules, expression by recombinant cells, receptor-mediated endocytosis. See e.g., Wu and Wu (1987) J. Biol. Chem. 262:4429-4432 for construction of a therapeutic nucleic acid as part of a retroviral or other vector, etc. Methods of delivery include but are not limited to intra-arterial, intra-muscular, intravenous, intranasal and oral routes. In a specific embodiment, it may be desirable to administer the pharmaceutical compositions of the invention locally to the area in need of treatment; this may be achieved by, for example, and not by way of limitation, local infusion during surgery, by injection or by means of a catheter.

Solutions containing the agent(s) can be prepared in suitable diluents such as water, ethanol, glycerol, liquid polyethylene glycol(s), various oils, and/or mixtures thereof, and others known to those skilled in the art.

The pharmaceutical forms of the agent(s) suitable for injectable use include sterile solutions, dispersions, emulsions, and sterile powders. The final form must be stable under conditions of manufacture and storage. Furthermore, the final pharmaceutical form must be protected against contamination and must, therefore, be able to inhibit the growth of microorganisms such as bacteria or fungi. A single intravenous or intraperitoneal dose can be administered. Alternatively, a slow long term infusion or multiple short term daily infusions may be utilized, typically lasting from 1 to 8 days. Alternate day or dosing once every several days may also be utilized.

Sterile, injectable solutions are prepared by incorporating a compound in the required amount into one or more appropriate solvents to which other ingredients, listed above or known to those skilled in the art, may be added as required. Sterile injectable solutions are prepared by incorporating the compound in the required amount in the appropriate solvent with various other ingredients as required. Sterilizing procedures, such as filtration, then follow. Typically, dispersions are made by incorporating the compound into a sterile vehicle which also contains the dispersion medium and the required other ingredients as indicated above. In the case of a sterile powder, the preferred methods include vacuum drying or freeze drying to which any required ingredients are added.

In all cases the final form, as noted, must be sterile and must also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

Prevention or inhibition of growth of microorganisms in the formulations may be achieved through the addition of one or more antimicrobial agents such as chlorobutanol, ascorbic acid, parabens, thermerosal, or the like. It may also be preferable to include agents that alter the tonicity such as sugars or salts.

The following examples are intended to illustrate but not limit the invention. The content of these experiments have been reported in Serenello et al. (2010) J. Biol. Chem. 285(2):845-854, incorporated by reference in its entirety.

### Example 1

### Experimental Procedures

HCV Constructs

The genotype 2a HCV constructs, pJFH1 (produces infectious virus particles), replicative-null pJFH1-GND, and subgenomic pSgJFH1-Luc (contains a luciferase reporter gene), are described elsewhere (23, 24). Subgenomic HCV replicons are bicistronic constructs that express only the nonstructural proteins of HCV under the control of encephalocarditis virus IRES; neomycin resistance or firefly luciferase gene is under the control of the HCV IRES (15, 22, 24). These replicons support HCV RNA replication but no virus is formed in cell culture. Huh7 cell clones (SgPC2 cells, Clone B) supporting continuous replication of subgenomic HCV replicon of genotype 1b (Con1 sequence) were also used (17, 22).

### RNA Transfection, Infection, and Cell Culture

The in vitro transcription, and transfection of HCV RNA, and Huh7 human hepatoma cell culture were performed as previously described (17). For experiments involving stable clones, cells were cultured in medium supplemented with 0.4-0.5 mg/mL G418, and G418 was removed from cell culture medium one day prior to cell treatments, which were performed as described in Results. For the in vitro infectivity assays, 2 ml of the extracellular medium from JFH1 RNA-transfected cells was used to inoculate naive Huh7 or Huh7.5 cells with 3 ml of fresh medium, as described (23, 25). Treatments were initiated 24 hours after infection and the cells were harvested after another 24 or 48 hrs.

### Northern Blot Analysis

Intracellular RNA extraction and northern blots were carried out, as described (16, 17). DNA probes were prepared from nucleotides (“nt.”) 4128-8273 or 358-2816 of JFH1, generated with ScaI and ApaL I, respectively, or nt. 3669 to 6016 of the Con1 subgenomic replicons. Images were quantified by densitometry, using Optiquant Cyclone 4.00 (Perkin Elmer), and data were normalized by glyceraldehyde 3-phosphate dehydrogenase (GAPDH) mRNA content.

Quantitative Real Time Reverse Transcriptase-Polymerase Chain Reaction (qRT-PCR)

The total intracellular RNA was obtained from cells using Trizol (Invitrogen). To obtain extracellular HCV RNA, cell culture medium samples were first treated with RNase A (100 μg/ml) for 30 min at room temperature, then RNA was extracted using Trizol LS and glycogen as a carrier. HCV RNA was quantified by qRT-PCR as described (17, 23). For JFH1, the primer sequences were 5′ TCTGCGGAACCGGTGAGTA 3′ (nt 146 to 164; forward, SEQ ID NO.: 1), and 5′ TCAGGCAGTACCACAAGGC 3′ (nt 277 to 295; reverse, SEQ ID NO.: 2), and the sequence of the fluorogenic probe, labeled with 6-FAM and TAMRA (Biosearch Technologies, Inc.), was 5′ CCAGTCTTCCCGGCAATTCCG 3′ (nt 168 to 188, SEQ ID NO.: 3). The primer and probe sequences for qRT-PCR analysis of Con1 RNA's were described previously (17). Standard curves were generated using in vitro-transcribed HCV RNA's. Intracellular HCV RNA levels were normalized by 18 S rRNA or GAPDH mRNA.

### Western Blot Analysis

Cells were sonicated in Laemmli buffer, and proteins were separated on 7-20% Tris-glycine gel (Invitrogen) or 12.5% SDS-polyacrylamide gel and western blotted, using mouse monoclonal anti-NS5A (Biodesign, Inc.), goat anti-actin, and the corresponding horseradish peroxidase-conjugated secondary antibodies (Santa Cruz Laboratories) with an ECL Plus kit (Amersham Biosciences). Images were obtained and quantified, using 152000R (Kodak).

### Luciferase Assays

After various treatments, SgJFH1 Luc RNA-transfected cells were lysed with 1× Reporter Lysis Buffer, and the luciferase activity was determined using Luciferase Reporter Assay Kit (Promega Corp.) (24). Luciferase activities were normalized by total protein content, determined with bicinchoninic acid (BCA) assay kit from Pierce.

### In Vitro HCV Replication Assay

In vitro replication assay was carried out according to the modified method of Ali et al., as previously described (16, 17, 26). Briefly, cytoplasmic lysates were prepared, and the replication was allowed to proceed for 1 hr at 30° C. in the presence of α-³²P-CTP and actinomycin D. Then, RNA products were analyzed on a 1% formaldehyde agarose gel, which was subsequently analyzed, using Optiquant Cyclone 4.00 (Perkin Elmer).

### NADH/NAD+ and ATP Assays

NADH and NAD+ levels were determined by enzymatic NADH recycling assay, using the NAD+/NADH Quantification Kit from Biovision, per manufacturers recommendations with an optional filtration step that used Microcon YM-10 (Millipore). Total ATP content was measured using Somatic Cell ATP Assay Kit from Sigma-Aldrich. The data were normalized by total protein content, determined with BCA assay kit from Pierce.

### Statistics

Data were analyzed using Student's t test or one-way analysis of variance, followed by post-hoc comparisons, using SigmaStat 3.1 (Jandel Scientific). A p value≦0.05 was considered significant. Data are presented as means±standard error of the mean (SEM) of several independent experiments. All experiments were repeated two to five times.

### Results

### Ethanol Increases the Complete Replication of HCV at Physiological Concentrations

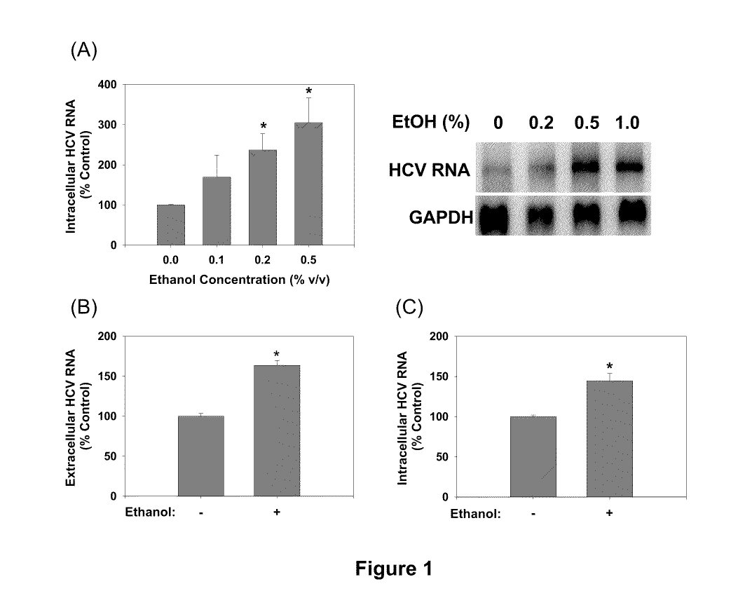

To examine whether ethanol increased the complete replication of HCV, positive-sense genomic JFH1 RNA was produced by in vitro transcription, using T7 RNA polymerase, and transfected into Huh7 human hepatoma cells. Then, the transfected cells were exposed to 0-1.0% (v/v; 0-172 mM) ethanol once daily with a change of medium each day for 48 hrs. Then, the cells and the cell culture medium were harvested and analyzed for intracellular and RNase A-resistant extracellular HCV RNA's by a combination of Northern blots and qRT-PCR. Ethanol significantly increased the intracellular JFH1 HCV RNA levels to 237±40 and 305±61% of untreated controls at 0.2 and 0.5% concentrations, respectively (P<0.05) (FIGS. 1A and 1B). The increases were less pronounced at 24 hrs. Extracellular HCV RNA was also significantly elevated with the ethanol treatment, indicating increased virus secretion (FIG. 1B). Next, whether virus-infected cells responded similarly to ethanol treatment with elevated HCV RNA was examined. It was found that ethanol also increased HCV RNA in Huh7 cells infected with cell culture-generated 0.2% JFH1 virion (FIG. 1C). JFH1 GND mutant, which harbors a critical mutation (GDD:GND) in NS5B, the viral polymerase, did not replicate or generate infectious virus particles, as expected. These concentrations of ethanol did not induce any cytotoxicity, as assessed by cell morphology and measuring the cellular ATP content (data not shown). The 0.2 ethanol, equivalent to blood alcohol concentration of 34.4 mM, that significantly enhanced HCV replication is approximately twice the legal limit for driving under the influence (DUI) in many countries, including the U.S. The 0.5% ethanol lies in the toxic range but can also be achieved physiologically, particularly in chronic alcohol users. In addition, ethanol is volatile and the amount that remains would be significantly less than what was added to the cell culture medium (27). These data, therefore, suggest that ethanol can enhance complete HCV replication, at physiological attainable concentrations.

### Ethanol Enhances HCV RNA Replication of Genotypes 2a and 1b

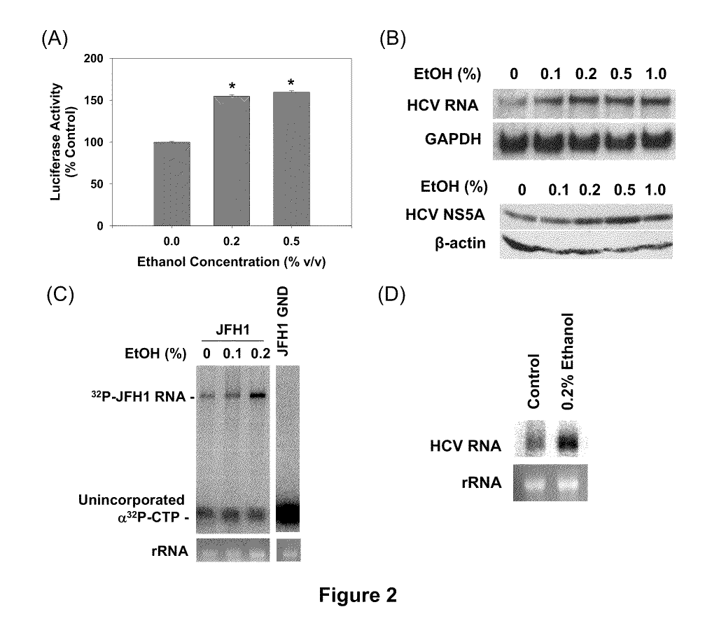

Previously, ethanol was shown to elevate HCV RNA content in Huh7 cells that supported subgenomic HCV RNA replication without virus production (9, 13, 14). To test whether the JFH1 RNA replication was also affected by ethanol, Huh7 cells were transfected with JFH1 SgJFH1-Luc RNA and exposed the cells to ethanol for 48 hours. Then, HCV replication was monitored by measuring the firefly luciferase activity (24). Ethanol increased the luciferase activity in these cells, suggesting that the JFH1 RNA genome replication was affected (FIG. 2A).

Genotype 2a HCV infection is found globally, with the prevalence ranging from less than 2 to about 30% depending on the geographical region (28, 29). However, as the most prevalent HCV genotype is genotype 1, these experiments were also repeated, using Con1 subgenomic replicon RNA of genotype 1b (17, 22). Again, significant increases in the genotype 1b HCV RNA could be demonstrated with 0.1 to 1% ethanol (FIG. 2B). Similar increases in the HCV NS5A protein content could be shown by Western blots (FIG. 2B, bottom panel).

To confirm that the rate of the HCV RNA genome replication is accelerated by ethanol, the activity of the HCV RNA replication complex was measured. JFH1-transfected cells were exposed to ethanol for 5 hrs and then, the cytoplasmic lysates, containing the HCV replication complex, were isolated. Then, the in vitro RNA replication assay was performed in the presence of α-³²P-labeled CTP and actinomycin D, as previously described (17). JFH1 GND cell lysates were used as a negative control. As shown in FIG. 2C, JFH1 cell extracts produced a single ³²P-labeled RNA band of ˜9.5 kb, corresponding to the expected size of the HCV RNA, indicating active viral RNA replication, whereas the JFH1 GND extracts did not. (FIG. 2C) Ethanol significantly increased the rate of HCV RNA replication at 5 hrs (FIG. 2C) but no significant increase was detected at ½, 1, or 3 hrs (data not shown). Ethanol also accelerated the in vitro replication rate of Con1 strain (FIG. 2D). On the other hand, ethanol did not increase the HCV IRES activity, as assessed by the HCV IRES activity assay, using pRL-HL (data not shown) (30). The data suggests that ethanol increases the rate of HCV RNA replication without directly enhancing the translation rate of the HCV polyprotein, at least when these processes are evaluated separately. Therefore, increases in the NS5A protein content with ethanol (FIG. 2B) is likely to have resulted from increased levels of the viral RNA template for translation.

CYP2E1 is present in Huh7 Cells at Levels Comparable to Human Liver

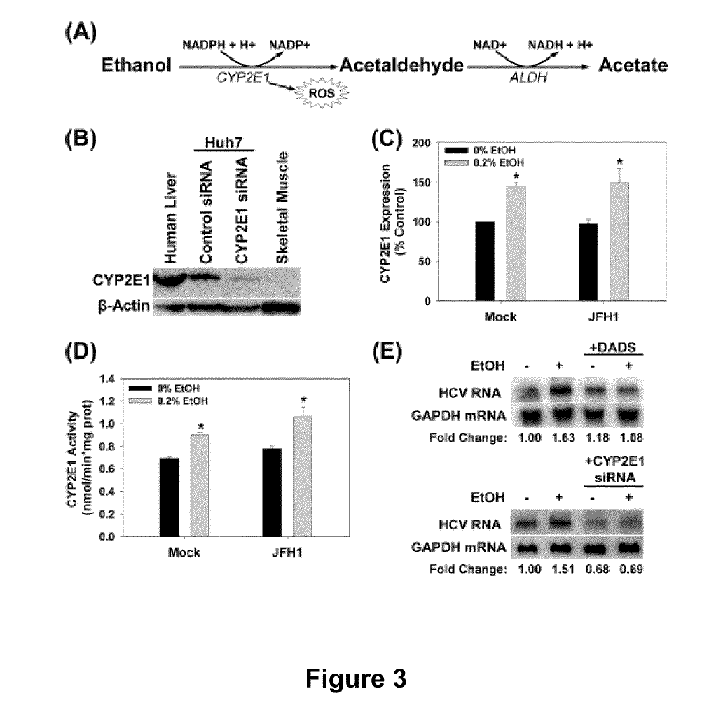

To confirm that ethanol metabolism is intact in the system, the Huh7 cells were analyzed for the expression of CYP2E1. The Huh7 cells were found to have CYP2E1 levels comparable to human liver (FIG. 3A). CYP2E1 expression could also be enhanced in JFH1 cells by daily treatment with 0.2% (v/v) ethanol for 48 hrs (FIG. 3C). This enhanced expression of CYP2E1 could be maintained for at least two weeks with daily ethanol treatments (data not shown). Furthermore, ethanol increased the NADH/NAD+ ratio by 76.2±6.1% within 3 hrs (P<0.05). These data suggest that ethanol is being metabolized by these cells and that an ectopic expression of CYP2E1 would be unnecessary for these cells.

Acetaldehyde, Rather than ROS, Increases the Replication of HCV

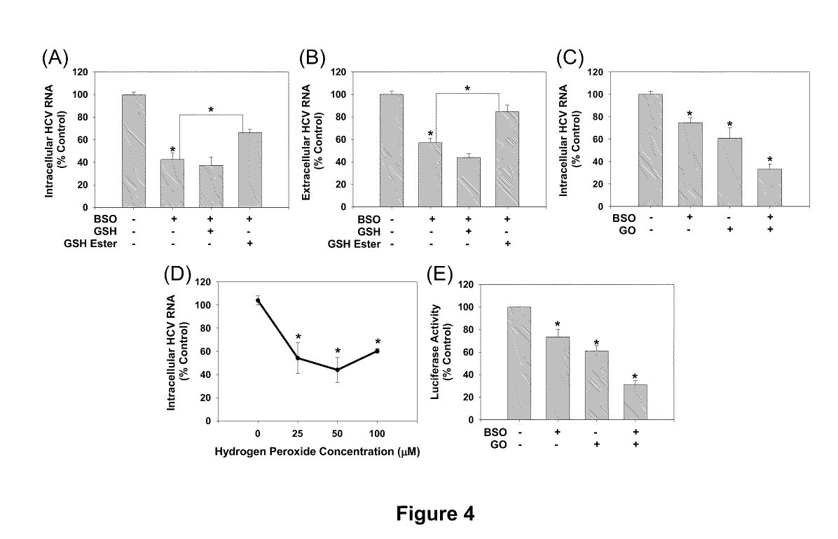

Hepatic ethanol, particularly CYP2E1-mediated, metabolism is known to generate ROS (31). To uncover the mechanism by which ethanol promotes HCV RNA replication, it was first evaluated whether ROS had similar effects on HCV as ethanol. First, to examine the effects of endogenously generated ROS, L-buthionine S,R-sulfoximine (BSO) was used. BSO depletes glutathione (GSH), a major endogenous antioxidant, by inhibiting its de novo synthesis and therefore, would amplify the effects of endogenous ROS, generated during normal cellular metabolism and in response to HCV (5). BSO decreases intracellular GSH content by approximately 80±12% in Huh7 cells (P<0.05). It was found that BSO significantly decreased both intracellular and extracellular JFH1 RNA levels (FIGS. 4A and B). GSH ethyl ester, which enters cells and is cleaved by cellular esterases to generate GSH inside cells (i.e., restores intracellular GSH, bypassing the inhibition of GSH biosynthesis by BSO), partially restored both intracellular and extracellular HCV RNA (FIGS. 4A and B). As a control, adding GSH, which is broken down into its amino acid constituents then gets taken up for intracellular de novo GSH synthesis (i.e., cannot bypass the BSO-inhibited step), could not restore the HCV RNA level in these cells, as expected (FIGS. 4A and B). The data suggests that BSO decreases HCV titer specifically by depleting GSH.

To examine the effects of the exogenous ROS, JFH1 RNA-transfected cells were incubated with 0.25 mU/mL of glucose oxidase (GO), which produces H₂O₂ extracellularly through an enzymatic reaction in the presence of glucose, mimicking inflammation. GO decreased the intracellular JFH1 RNA by 30±8% (P<0.05) and exacerbated the suppression of HCV RNA by BSO, indicating that both endogenous and exogenous ROS suppress HCV replication (FIG. 4C). In addition, JFH1 RNA levels decreased with 25, 50, and 100 μM H₂O₂ (FIG. 4D). Treating cells with BSO alone or with GO likewise suppressed the subgenomic JFH1 RNA replication, and GO exacerbated the suppression by BSO (FIG. 4E). These cell treatments did not induce cytotoxicity, as determined by the ATP assay (data not shown). The suppression of HCV RNA by H₂O₂ also occurred at subtoxic concentrations. The highest concentration of H₂O₂ used (100 μM) did show some cytotoxicity in the JFH1 cells but even at this concentration, JFH1 RNA was decreased compared to the control (0 μM H₂O₂, P<0.05, FIG. 4D), and there was no significant difference in the level of JFH1 RNA at 100 μM H₂O₂ versus 25 and 50 μM H₂O₂ (P<0.05, FIG. 4D). These observations are consistent with previous findings from this laboratory that showed a rapid suppression of Con1 subgenomic and H77c/ConI-hybrid-genomic HCV RNA replication by exogenous as well as endogenous ROS (16, 17).

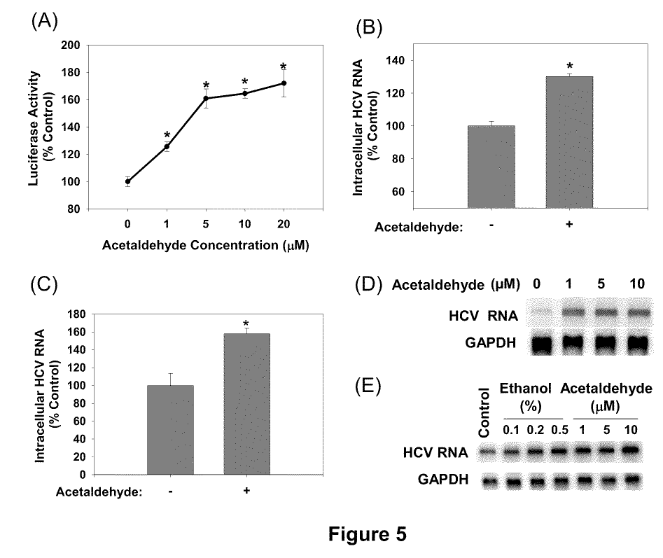

It was next evaluated whether acetaldehyde, a major product of ethanol metabolism, could potentiate HCV replication in the virus-producing as well as the non-virus-producing subgenomic replicon systems. Acetaldehyde, at physiologically relevant concentrations (32, 33), significantly increased the HCV RNA content in both the non-virus producing and virus-producing JFH1 cells (FIGS. 5A and 5B). Infecting naive cells with virus-containing medium and then treating with acetaldehyde also led to significant increases in HCV replication (FIG. 5C). To examine whether acetaldehyde had similar effects on genotype 1b HCV (SgCon1-Neo), SgPC2 cells were treated with acetaldehyde and analyzed for changes in the HCV RNA replication. Acetaldehyde likewise elevated the HCV RNA level in these cells (FIG. 5D). Thus, acetaldehyde is sufficient to potentiate HCV replication of both genotypes 1b and 2a, as it has been observed with ethanol (FIGS. 1 and 2). Another Con1 HCV subgenomic replicon cell clone, Clone B, derived at another laboratory (22), also responded similarly to ethanol and acetaldehyde, indicating that the response is not specific to the cell clones (FIG. 5E).

### Isopropyl Alcohol and Acetone Also Potentiate HCV Replication, the Role of NADH/NAD+

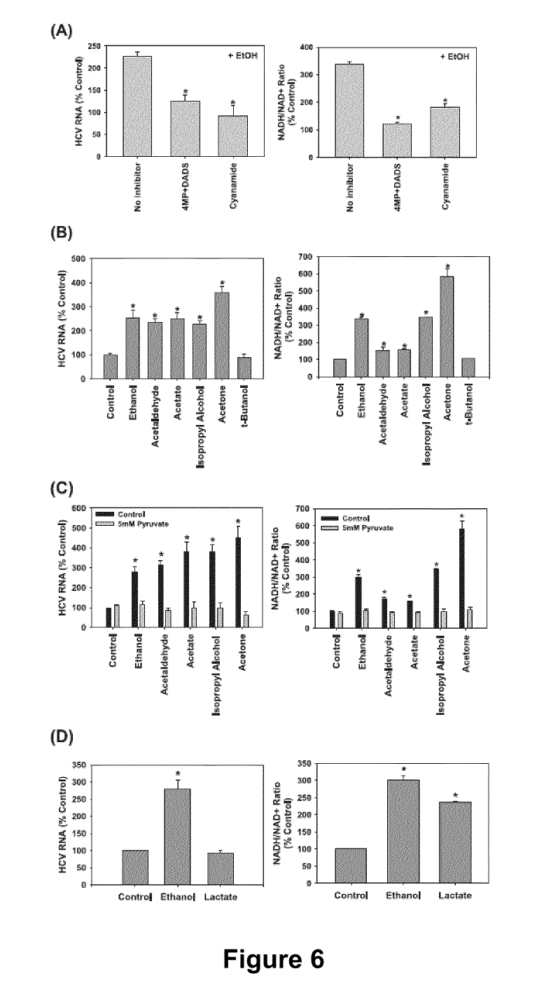

Applicants continued to investigate whether acetaldehyde itself or products of acetaldehyde metabolism are critical for the potentiation of HCV replication by ethanol by inhibiting aldehyde dehydrogenase with cyanamide (see FIG. 3A). Cyanamide suppressed the potentiation of HCV replication by ethanol just as inhibiting the first step of ethanol metabolism with 4-methylpyrazole (4 MP) and DADS did, suggesting that it is not acetaldehyde itself but a downstream product of acetaldehyde metabolism that increases HCV replication (FIG. 6A).

Acetaldehyde metabolism by aldehyde dehydrogenase generates NADH and acetate (FIG. 3A). To determine the potential role of NADH, Applicants first evaluated the effects of isopropyl alcohol. Isopropyl alcohol (0.2%, v/v) increases the levels of NADH like ethanol but generates acetone instead of acetaldehyde. To Applicants' surprise, isopropyl alcohol also increased the HCV RNA level (FIG. 6B) (26). Both isopropyl alcohol and ethanol increased NADH/NAD+ ratio in these cells, as expected (FIG. 6B). In contrast, tert-butanol did not elevate HCV replication or the NADH/NAD+ ratio.

Moreover, Applicants found that acetate itself increased the level of HCV RNA as treating cells with acetone also did. In addition, ethanol, acetaldehyde, acetate, isopropyl alcohol, and acetone all showed corresponding increases in NADH/NAD+ ratios (FIG. 6B) (4, 27). The NADH/NAD+ ratios were positively correlated with HCV RNA content in all of these treatments (r=0.95, p<0.001). The suppression of HCV replication by cyanamide, 4 MP, and DADS was also associated with corresponding decreases in the NADH/NAD+ ratios (data not shown). Therefore, changes in HCV replication paralleled the changes in the NADH/NAD+ ratio, produced by these treatments.

Then, Applicants examined whether increased NADH/NAD+ ratio was required for the potentiation of HCV replication by ethanol and these other agents. Pyruvate, which re-oxidizes cytosolic NADH to NAD+, completely abrogated the increases in HCV replication and NADH levels during ethanol, acetaldehyde, acetate, isopropyl alcohol, and acetone treatments. Methylene blue, which also oxidizes NADH, had similar effects on HCV as pyruvate (data not shown). In contrast, lactate, which produces NADH in the cytosol independent of ethanol, increased NADH levels to 235.9±11.9% (p<0.05) of the control level but had little to no effect on HCV replication (data not shown). Together, these data indicate that whereas an alteration of cellular NADH/NAD+ levels seems necessary for the ethanol-induced increases in HCV replication, elevated NADH/NAD+ may not be sufficient to increase HCV replication.

### The Potentiation of HCV Replication by Ethanol Requires Lipogenesis

NADH has diverse functions in the cell, and one of these functions includes modulation of lipid metabolism. For example, NADH can inhibit mitochondrial β-oxidation and increase fatty acid synthesis (35). It is well-established that ethanol modulates fatty acid metabolism in part through NADH, and that this plays an important role in the development of steatosis in the alcoholic liver (35). Acetate and acetone would generate acetyl-CoA, which also drives lipogenesis (51, 35). Furthermore, cholesterol metabolism and fatty acid biosynthesis are important in HCV RNA replication (53). Lovastatin and fluvastatin, which are competitive inhibitors of 3-hydroxy-3-methyl-glutaryl-CoA reductase, and 5-(tetradecyloxy)-2-furoic acid (TOFA) and cerulenin, which inhibits fatty acid biosynthesis, have been shown to suppress the basal level of HCV replication (53, 54). Therefore, Applicants next examined whether the potentiation of HCV RNA replication by above agents might be inhibited by modulators of lipid metabolism.

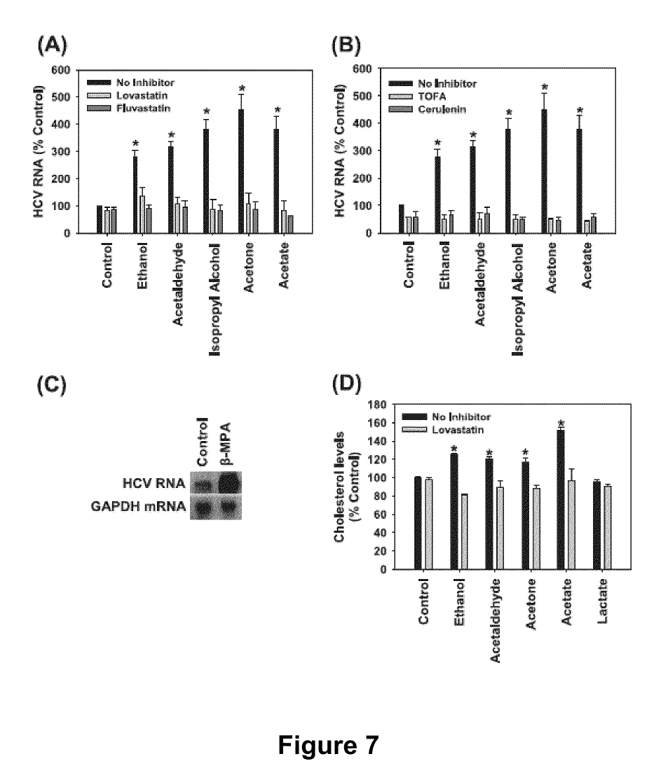

Lovastatin, fluvastatin, TOFA, and cerulenin almost completely inhibited the potentiation of HCV RNA replication by ethanol, acetaldehyde, isopropyl alcohol, acetone, and acetate (FIGS. 7, A and B). In addition, inhibiting n-oxidation of fatty acids with β-mercaptopropionic acid caused a 15.2±1.7-fold (p<0.01) increase in HCV replication in these cells (FIG. 7C). Furthermore, ethanol, acetaldehyde, acetone, and acetate treatments increased the total intracellular cholesterol content, which was attenuated by lovastatin (FIG. 7D). Lactate, which increased NADH/NAD+ without increasing HCV replication, had no significant effect on cholesterol levels (FIG. 7D). The data suggest that the elevation of HCV replication by ethanol, acetaldehyde, acetone, and acetate is mediated by increases in intracellular cholesterol and can be abrogated by the inhibition of cholesterol or fatty acid biosynthetic pathways.

### Discussion

High HCV titer is directly associated with the development and progression of liver diseases (41). In addition, ethanol consumption, high BMI, and high viral titer are strongly associated with poor response to anti-HCV therapy (44). Therefore, the increased HCV replication observed with physiological levels of ethanol and acetaldehyde is likely to represent an important mechanism of the pathological interactions between HCV and ethanol in liver diseases and at least partly explain the negative effects of ethanol on interferon-α therapy. Previously, ethanol has been shown to suppress the antiviral function of interferon-α by interfering with the JAK-STAT signaling pathway (46); however, this is not likely to explain the potentiation of HCV replication observed with ethanol because HCV effectively suppresses the type I interferon response in these Huh cells. Additionally, ethanol and acetaldehyde could increase HCV replication in RIG-1-defective Huh7.5 cells (FIG. 5; also, data not shown) (47, 50).

Previously, it has been suggested that some of the key ethanol metabolizing enzymes might not be expressed in Huh7 cells (46). Indeed, it was also found that alcohol dehydrogenase I is decreased in the Huh7 cells compared to human liver (data not shown). However, CYP2E1 is expressed in the cells at levels comparable to human liver, and CYP2E1 expression could be enhanced by ethanol in JFH1 cells (FIG. 3). In addition, ethanol and acetaldehyde elevated NADH/NAD+ ratio, indicating that ethanol is being metabolized by the cells. Indeed, even if the cells do not have all of the normal ethanol metabolizing enzymes, the discovery that acetaldehyde and acetate can enhance HCV replication is significant, as they bypass these reactions.

As alcohol dehydrogenase is less prone to ROS generation than CYP2E1, it may also be speculated that ethanol would cause even greater potentiation of HCV replication in cells that express normal levels of this enzyme. Previous study by Zhang et al., using various chemical inhibitors of ethanol metabolism, suggested that ethanol metabolites were involved in the potentiation of subgenomic HCV RNA replication by ethanol (9). The data are in agreement with this study and suggest that ethanol and acetaldehyde also directly enhance HCV replication in the context of the complete viral replication cycle. In terms of the mechanism, Applicants found that isopropyl alcohol, acetone, and acetate also increase HCV replication, and increased NADH/NAD+ ratio was required for the potentiation of HCV replication by ethanol, acetaldehyde, as well as isopropyl alcohol, acetone, and acetate. In contrast, t-butanol, a tertiary alcohol that is poorly metabolized by humans and does not increase the NADH/NAD+ ratio, did not elevate HCV replication, as predicted by Applicants' model. The NADH/NAD+ ratio in ethanol-treated cells was decreased by cyanamide, suggesting that NADH is generated downstream of acetaldehyde (FIG. 3A). Acetate, the downstream metabolite of acetaldehyde, was previously considered inert but there is evidence that it can be converted to acetyl-CoA and other metabolic intermediates by mammalian cells (31, 35). Isopropyl alcohol is known to be metabolized into acetone and possibly other ketone bodies that can also be converted to acetyl-CoA (51). The mechanism by which isopropyl alcohol increases the NADH/NAD+ ratio in our system is unclear and may involve residual ADH or hitherto uncharacterized enzyme activity that is induced by HCV.

In terms of how NADH increases HCV replication, NADH plays key roles in cellular bioenergetics and can modulate fatty acid synthesis as well as suppress beta-oxidation (31, 35). Applicants were interested in the potential involvement of lipids because HCV replicates in cholesterol-rich compartments in the cell, and cholesterol and fatty acid metabolism have been shown to be important for HCV replication (53). Specifically, cholesterol metabolism increases basal HCV replication by the geranylgeranylation of FBL2 (53). Applicants found that inhibiting the host mevalonate pathway with statins and fatty acid synthesis with TOFA or cerulenin blunted the potentiation of HCV replication by ethanol, acetaldehyde, isopropyl alcohol, acetone, and acetate, whereas inhibiting beta-oxidation dramatically increased HCV replication (FIG. 7). In addition, the potentiation of HCV replication by these agents was accompanied by an increase in the intracellular cholesterol content, which was attenuated by lovastatin (FIG. 7D). Regarding potential effects of NADH on the ATP, overall ATP levels were not significantly perturbed in these cells by ethanol or other treatments (data not shown), suggesting that ATP is not likely to explain the effects that ethanol had on HCV. In fact, ethanol also increased the rate of HCV replication in the in vitro replication assay (FIGS. 2C and 2D) which was performed in the presence of excess ATP. Taken together, these data indicate that the potentiation of HCV replication by ethanol, acetaldehyde, acetate, isopropyl alcohol, and acetone ultimately requires host lipid metabolism and is sensitive to lipid modulators, which points to potential targets for therapy. The concentrations of lovastatin and fluvastatin used here are higher than the doses used clinically to treat hypercholesterolemia. However, it is possible that statins, if used in combination with antivirals or other lipid modulators, will help control HCV replication, particularly in chronic alcoholics who show resistance to standard anti-HCV therapy (52). It is also interesting to note that the concentrations of acetone that enhanced HCV replication in this study are physiological levels that can be attained during metabolic dysfunction such as diabetes and during starvation (51), and HCV infection can lead to insulin resistance (49). In addition, acetate, which increased HCV replication at micromolar to millimolar concentrations in this study (data not shown), is used in hemodialysis.

Interestingly, increasing the NADH/NAD+ ratio with lactate was not sufficient to increase HCV replication, suggesting that other factors may also play a role (FIG. 7D). Lactate also did not increase the intracellular cholesterol level. These results are consistent with an important role of cholesterol in the regulation of HCV replication. The data also indicate that even though ethanol and lactate both increase the NADH/NAD+ ratio, ethanol is more lipogenic than lactate in these cells. The reason for these differences is unclear but it might be explained at least in part by the fact that ethanol can inhibit citric acid cycle as well as gluconeogenesis, which may cause acetate/acetyl-CoA produced by ethanol metabolism to be shunted more toward the lipogenic pathways, whereas these processes are likely to be stimulated by lactate (55). Ethanol can also decrease the total oxidation of fatty acids to CO2, and increase the breakdown of glycogen, which may further drive lipogenesis in these cells (55-57). Further investigation into these effects will be beneficial to understanding how different metabolic conditions would affect HCV replication in hepatocytes.

Recently, McCartney et al. reported an elevation of HCV RNA by ethanol in Huh7 replicon cells, transfected with CYP2E1; the effect could be suppressed by NAC, leading to the conclusion that the increase was due to ROS generation by CYP2E1 (13). In contrast, it has been consistently found that ROS suppresses HCV replication while GSH, NAC, and vitamin E tend to counter this suppression (16-18, 20, 21) (FIG. 4). In particular, the BSO studies clearly demonstrate that endogenous ROS are sufficient to suppress HCV replication in cell culture (16, 17). The reason for this discrepancy is unclear. However, CYP2E1 generates acetaldehyde as well as ROS, both of which can react with thiols, such as cysteine and GSH (35). NAC is a precursor of cysteine, which is used to synthesize GSH. NAC can also have other effects, including alteration of the pH and acting as a pro-oxidant. Therefore, the study by McCartney et al. does not differentiate whether the potentiation of HCV replication by ethanol is due to ROS, acetaldehyde, or other variables (13). Indeed, other studies also showed an enhancement of HCV RNA replication by antioxidants (e.g., vitamins E and C) and a suppression of viral replication by lipid peroxidation products and ROS (18, 20, 21), and this suppression is likely to involve calcium and the dissociation of HCV replication complex from the membranes (16, 17). In terms of the mechanism of how ethanol, acetaldehyde, isopropanol, and acetone increase HCV replication, all of these agents elevated the NADH/NAD+ ratio. NADH plays key roles in cellular bioenergetics and can modulate fatty acid synthesis and suppress β-oxidation (31, 35). Acetaldehyde and isopropanol are also metabolized to acetate, acetone, and possibly other ketone bodies, all of which can be converted to acetyl-CoA (31, 35, 51). It was found that increased NADH is required for the potentiation of HCV replication by ethanol but not sufficient to increase HCV replication, suggesting that other factors, such as acetyl-CoA, are also likely to play a role. Furthermore, inhibiting the host mevalonate pathway with lovastatin and fatty acid synthesis with TOFA blunted the potentiation of HCV replication by these agents, while inhibiting β-oxidation dramatically increased HCV replication. Therefore, the data suggest that the potentiation of HCV replication by ethanol, acetaldehyde, acetate, isopropanol, and acetone ultimately requires host lipid metabolism and is sensitive to lipid modulators, which points to potential targets for therapy. In contrast, ATP levels were not significantly perturbed in these cells by ethanol or other treatments (data not shown), suggesting that ATP is not likely to explain the effects that ethanol had on HCV. In fact, ethanol also increased the rate of HCV replication in the in vitro replication assay (FIG. 2) which is performed in the presence of excess ATP. The concentrations of lovastatin used here is higher than the doses used clinically to treat hypercholesterolemia. However, statins, used in combination with antivirals or other lipid modulators, may help control HCV replication, particularly in chronic alcoholics who show resistance to standard anti-HCV therapy (52).

Finally, the concentrations of acetone that enhanced HCV replication in this study are physiological levels that can be attained during metabolic dysfunction such as diabetes and during starvation (51). HCV infection can lead to metabolic conditions such as insulin resistance (49). Therefore, increased levels of acetone and possibly other ketone bodies may accelerate HCV replication during virus-induced insulin resistance. It is also important to note that acetate, which increased HCV replication at micromolar concentrations, is used in millimolar concentrations for hemodialysis. Therefore, in this study, it is shown that physiological levels of ethanol, acetaldehyde, and acetone promote HCV replication in the context of the complete HCV replication, and that the response is likely mediated by the modulation of host lipid metabolism. The potent effect that acetone has on HCV replication may have special significance for patients with HCV-induced insulin resistance. Further study into the precise mechanisms of this regulation may lead to the development of novel treatments that target both the virus and its pathogenic interactions with ethanol in chronic hepatitis C patients.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

### REFERENCES

1. Chang T K, Crespi C L, Waxman D J. Spectrophotometric analysis of human CYP2E1-catalyzed p-nitrophenol hydroxylation. Methods Mol Biol 1998; 107:147-152.
2. Jamal M M, Morgan T R. Liver disease in alcohol and hepatitis C. Best Pract Res Clin Gastroenterol 2003; 17:649-662.
3. Sata M, Fukuizumi K, Uchimura Y, Nakano H, Ishii K, Kumashiro R, Mizokami M, et al. Hepatitis C virus infection in patients with clinically diagnosed alcoholic liver diseases. J Viral Hepat 1996; 3:143-148.
4. Monto A, Alonzo J, Watson J J, Grunfeld C, Wright T L. Steatosis in chronic hepatitis C: Relative contributions of obesity, diabetes mellitus, and alcohol. Hepatology 2002; 36:729-736.
5. Seronello S, Sheikh M Y, Choi J. Redox regulation of hepatitis C in nonalcoholic and alcoholic liver. Free Radic Biol Med 2007; 43:869-882.
6. Carriere M, Rosenberg A R, Conti F, Chouzenoux S, Terris B, Sogni P, Soubrane O, et al. Low density lipoprotein receptor transcripts correlates with liver hepatitis C virus RNA in patients with alcohol consumption. J Viral Hepat 2006; 13:633-642.
7. Romero-Gomez M, Grande L, Nogales M C, Fernandez M, Chavez M, Castro M. Intrahepatic hepatitis C virus replication is increased in patients with regular alcohol consumption. Dig Liver Dis 2001; 33:698-702.
8. Oshita M, Hayashi N, Kasahara A, Hagiwara H, Mita E, Naito M, Katayama K, et al. Increased serum hepatitis C virus RNA levels among alcoholic patients with chronic hepatitis C. Hepatology 1994; 20:1115-1120.
9. Zhang T, Li Y, Lai J P, Douglas S D, Metzger D S, O'Brien C P, Ho W Z. Alcohol potentiates hepatitis C virus replicon expression. Hepatology 2003; 38:57-65.
10. Tamai T, Seki T, Shiro T, Nakagawa T, Wakabayashi M, Imamura M, Nishimura A, et al. Effects of alcohol consumption on histological changes in chronic hepatitis C: a clinicopathological study. Alcohol Clin Exp Res 2000; 24:106 S-111S.
11. Pessione F, Degos F, Marcellin P, Duchatelle V, Njapoum C, Martinot-Peignoux M, Degott C, et al. Effect of alcohol consumption on serum hepatitis C virus RNA and histological lesions in chronic hepatitis C. Hepatology 1998; 27:1717-1722.
12. Cromie S L, Jenkins P J, Bowden D S, Dudley F J. Chronic hepatitis C: effect of alcohol on hepatitic activity and viral titre. J Hepatol 1996; 25:821-826.
13. McCartney E M, Semendric L, Helbig K J, Hinze S, Jones B, Weinman S A, Beard M R. Alcohol Metabolism Increases the Replication of Hepatitis C Virus and Attenuates the Antiviral Action of Interferon. J Infect Dis 2008; 198, 1766-1775.
14. Trujillo-Murillo K, Alvarez-Martinez O, Garza-Rodriguez L, Martinez-Rodriguez H, Bosques-Padilla F, Ramos-Jimenez J, Barrera-Saldana H, et al. Additive effect of ethanol and HCV subgenomic replicon expression on COX-2 protein levels and activity. Viral Hepat 2007; 14:608-617.
15. Moradpour D, Penin F, Rice C M. Replication of hepatitis C virus. Nat Rev Microbiol 2007; 5:453-463.
16. Choi J, Forman H J, Ou J H, Lai M M, Seronello S, Nandipati A. Redox modulation of the hepatitis C virus replication complex is calcium dependent. In: Free Radic Biol Med; 2006. p. 1488-1498.
17. Choi J, Lee K J, Zheng Y, Yamaga A K, Lai M M, Ou J H. Reactive oxygen species suppress hepatitis C virus RNA replication in human hepatoma cells. Hepatology 2004; 39:81-89.
18. Huang H, Chen Y, Ye J. Inhibition of hepatitis C virus replication by peroxidation of arachidonate and restoration by vitamin E. Proc Natl Acad Sci USA 2007; 104:18666-18670.
19. Waris G, Turkson J, Hassanein T, Siddiqui A. Hepatitis C virus (HCV) constitutively activates STAT-3 via oxidative stress: role of STAT-3 in HCV replication. J Virol 2005; 79:1569-1580.
20. Yano M, Ikeda M, Abe K, Dansako H, Ohkoshi S, Aoyagi Y, Kato N. Comprehensive analysis of the effects of ordinary nutrients on hepatitis C virus RNA replication in cell culture. Antimicrob Agents Chemother 2007; 51:2016-2027.
21. Kuroki M, Ariumi Y, Ikeda M, Dansako H, Wakita T, Kato N. Arsenic trioxide inhibits hepatitis C virus RNA replication through modulation of the glutathione redox system and oxidative stress. J Virol 2009; 83:2338-2348.
22. Blight K J, Kolykhalov A A, Rice C M. Efficient initiation of HCV RNA replication in cell culture. Science 2000; 290:1972-1974.
23. Wakita T, Pietschmann T, Kato T, Date T, Miyamoto M, Zhao Z, Murthy K, et al. Production of infectious hepatitis C virus in tissue culture from a cloned viral genome. Nat Med 2005; 11:791-796.
24. Kato T, Date T, Miyamoto M, Sugiyama M, Tanaka Y, Orito E, Ohno T, et al. Detection of anti-hepatitis C virus effects of interferon and ribavirin by a sensitive replicon system. J Clin Microbiol 2005; 43:5679-5684.
25. Blight K J, McKeating J A, Marcotrigiano J, Rice C M. Efficient replication of hepatitis C virus genotype 1a RNAs in cell culture. J Virol 2003; 77:3181-3190.
26. Ali N, Tardif K D, Siddiqui A. Cell-free replication of the hepatitis C virus subgenomic replicon. J Virol 2002; 76:12001-12007.
27. Eysseric H, Gonthier B, Soubeyran A, Bessard G, Saxod R, Barret L. There is not simple method to maintain a constant ethanol concentration in long-term cell culture: keys to a solution applied to the survey of astrocytic ethanol absorption. Alcohol 1997; 14:111-115.
28. Shin H R. Epidemiology of hepatitis C virus in Korea. Intervirology 2006; 49:18-22.
29. Marshall D J, Heisler L M, Lyamichev V, Murvine C, Olive D M, Ehrlich G D, Neri B P, et al. Determination of hepatitis C virus genotypes in the United States by cleavase fragment length polymorphism analysis. J Clin Microbiol 1997; 35:3156-3162.
30. Honda M, Kaneko S, Matsushita E, Kobayashi K, Abell G A, Lemon S M. Cell cycle regulation of hepatitis C virus internal ribosomal entry site-directed translation. Gastroenterology 2000; 118:152-162.
31. Cederbaum A I. Microsomal generation of reactive oxygen species and their possible role in alcohol hepatotoxicity. Alcohol Alcohol Suppl 1991; 1:291-296.
32. Peters T J, Ward R J, Rideout J, Lim C K. Blood acetaldehyde and ethanol levels in alcoholism. Prog Clin Biol Res 1987; 241:215-230.
33. Adachi J, Mizoi Y, Fukunaga T, Ogawa Y, Imamichi H. Comparative study on ethanol elimination and blood acetaldehyde between alcoholics and control subjects. Alcohol Clin Exp Res 1989; 13:601-604.
34. Clemens D L, Forman A, Jerrells T R, Sorrell M F, Tuma D J. Relationship between acetaldehyde levels and cell survival in ethanol-metabolizing hepatoma cells. Hepatology 2002; 35:1196-1204.
35. Lieber C S. Alcoholic fatty liver: its pathogenesis and mechanism of progression to inflammation and fibrosis. Alcohol 2004; 34:9-19.
36. Kapadia S B, Chisari F V. Hepatitis C virus RNA replication is regulated by host geranylgeranylation and fatty acids. Proc Natl Acad Sci USA 2005; 102:2561-2566.
37. Wang C, Gale M, Jr., Keller B C, Huang H, Brown M S, Goldstein J L, Ye J. Identification of FBL2 as a geranylgeranylated cellular protein required for hepatitis C virus RNA replication. Mol Cell 2005; 18:425-434.
38. Ye J, Wang C, Sumpter R, Jr., Brown M S, Goldstein J L, Gale M, Jr. Disruption of hepatitis C virus RNA replication through inhibition of host protein geranylgeranylation. Proc Natl Acad Sci USA 2003; 100:15865-15870.
39. Lonardo A, Adinolfi L E, Loria P, Carulli N, Ruggiero G, Day C P. Steatosis and hepatitis C virus: mechanisms and significance for hepatic and extrahepatic disease. Gastroenterology 2004; 126:586-597.
40. Bieche I, Asselah T, Laurendeau I, Vidaud D, Degot C, Paradis V, Bedossa P, et al. Molecular profiling of early stage liver fibrosis in patients with chronic hepatitis C virus infection. Virology 2005; 332:130-144.
41. Gretch D, Corey L, Wilson J, dela Rosa C, Willson R, Carithers R, Jr., Busch M, et al. Assessment of hepatitis C virus RNA levels by quantitative competitive RNA polymerase chain reaction: high-titer viremia correlates with advanced stage of disease. Infect Dis 1994; 169:1219-1225.
42. Mita E, Hayashi N, Hagiwara H, Ueda K, Kanazawa Y, Kasahara A, Fusamoto H, et al. Predicting interferon therapy efficacy from hepatitis C virus genotype and RNA titer. Dig Dis Sci 1994; 39:977-982.
43. Chayama K, Tsubota A, Kobayashi M, Okamoto K, Hashimoto M, Miyano Y, Koike H, et al. Pretreatment virus load and multiple amino acid substitutions in the interferon sensitivity-determining region predict the outcome of interferon treatment in patients with chronic genotype 1b hepatitis C virus infection. Hepatology 1997; 25:745-749.
44. Hosogaya S, Ozaki Y, Enomoto N, Akahane Y. Analysis of prognostic factors in therapeutic responses to interferon in patients with chronic hepatitis C. Transl Res 2006; 148:79-86.
45. Chen J, Clemens D L, Cederbaum A I, Gao B. Ethanol inhibits the JAK-STAT signaling pathway in freshly isolated rat hepatocytes but not in cultured hepatocytes or HepG2 cells: evidence for a lack of involvement of ethanol metabolism. Clin Biochem 2001; 34:203-209.
46. Plumlee C R, Lazaro C A, Fausto N, Polyak S J. Effect of ethanol on innate antiviral pathways and HCV replication in human liver cells. Virol J 2005; 2:89.
47. Gale M, Jr., Foy E M. Evasion of intracellular host defence by hepatitis C virus. Nature 2005; 436:939-945.
48. Kalapos M P. On the mammalian acetone metabolism: from chemistry to clinical implications. Biochim Biophys Acta 2003; 1621:122-139.
49. Sheikh M Y, Choi J, Qadri I, Friedman J E, Sanyal A J. Hepatitis C virus infection: molecular pathways to metabolic syndrome. Hepatology 2008; 47:2127-2133.
50. Blight K J, McKeating J A, Rice C M. Highly permissive cell lines for subgenomic and genomic hepatitis C virus RNA replication. J Virol 2002; 76:13001-13014.
51. Kalapos M P. Possible physiological roles of acetone metabolism in humans. Med Hypotheses 1999; 53:236-242.
52. Bader T, Fazili J, Madhoun M, Aston C, Hughes D, Rizvi S, Seres K, et al. Fluvastatin inhibits hepatitis C replication in humans. Am J Gastroenterol 2008; 103:1383-1389.
53. Ye J. PLoS Pathog 2007; 3, e108
54. Sagan S M, Rouleau Y, Leggiadro C, Supekova L, Schultz P G, Su Al and Pezacki J P. The influence of cholesterol and lipid metabolism on host cell structure and hepatitis C virus replication. Biochem Cell Biol 2006; 84, 67-79
55. Badawy A A. A Review of the Effects of Alcohol on Carbohydrate Metabolism. Alcohol Alcohol 1977; 12, 120-136
56. Adiels M, Boren J, Caslake M J, Stewart P, Soro A, Westerbacka J, Wennberg B, Olofsson S O, Packard C and Taskinen M R. Overproduction of VLDL₁ Driven by Hyperglycemia Is a Dominant Feature of Diabetic Dyslipidemia. Arterioscler Thromb Vasc Biol. 2005; 25, 1697-1703.
57. Towle H C. Metabolic Regulation of Gene Transcription in Mammals. J Biol. Chem. 1995; 270, 23235-23238.

What is claimed is:

1. A method for inhibiting replication of an RNA virus in a cell infected with the virus, wherein the cell is characterized as having been or concurrently being exposed to a physiologically relevant concentration of a compound selected from ethanol, acetate, isopropanol, acetaldehyde or acetone, comprising contacting the cell with an effective amount of one or more of an HMG-CoA reductase inhibitor mevalonate pathway inhibitor, statins, a fatty acid biosynthesis inhibitor TOFA (5-(Tetradecyloxy)-2-furoic acid), cerulenin, thyroxine to decrease NADH/NAD+ ratio or an agent promoting clearance of the compound from a cell, thereby inhibiting replication of the virus in the cell.

2. A method for inhibiting replication of an RNA virus in a cell infected with the virus, wherein the cell is characterized as having been or concurrently being exposed to a physiologically relevant concentration of a compound selected from ethanol, acetate, isopropanol, acetaldehyde or acetone, comprising contacting the cell with an effective amount of an agent selected from atovastatin, cerivastatin, fluvastatin, lovastatin, mevastatin, pitavastatin, pravastatin, rosuvastatin, simastatin, TOFA, cerulenin, thyroxine, bezafibrate, ciprofibrate, clofibrate, gemifibrozil and fenofibrate, thereby inhibiting replication of the virus in the cell.

3. A method for treating a subject infected with an RNA virus, wherein one or more cells in the subject is characterized as having been or concurrently being exposed to a physiologically relevant concentration of a compound selected from ethanol, acetate, isopropanol, acetaldehyde or acetone, comprising administering to the subject an effective amount of one or more of an HMG-CoA reductase inhibitor, a fatty acid biosynthesis inhibitor, thyroxine or an agent promoting clearance of the compound from a cell, thereby inhibiting replication of the virus in the cell.

4. A method for treating a subject infected with an RNA virus, wherein one or more cells in the subject are characterized as having been or concurrently being exposed to a physiological concentration of a compound selected from ethanol, acetate, isopropanol, acetaldehyde or acetone, comprising administering to the subject an effective amount of an agent selected from atovastatin, cerivastatin, fluvastatin, lovastatin, mevastatin, pitavastatin, pravastatin, rosuvastatin, simastatin, TOFA, cerulenin, thyroxine, bezafibrate, ciprofibrate, clofibrate, gemifibrozil and fenofibrate, thereby treating the subject.

5. The method of any one of claims 1-4, wherein the RNA virus is a positive sense single strand RNA virus.

6. The method of claim 5, wherein the positive sense single strand RNA virus is selected from a Yellow fever virus, a West Nile virus, a Dengue Fever virus or a hepatitis C virus (HCV).

7. The method of any one of claims 1-4, wherein the virus is HCV.

8. The method of any one of claims 1-4, wherein the method further comprises contacting the cell with an anti-RNA viral agent.

9. The method of claim 8, wherein the RNA virus is HCV and the anti-RNA viral agent is an anti-HCV agent.

10. The method of claim 9, wherein the anti-HCV agent is one which produces a subtoxic concentration of hydrogen peroxide.

11. The method of claim 10, wherein the anti-HCV agent is interferon, plerixafor, ribavirin, pegylated interferon-alpha-2a or pegylated interferon-alpha-2b.

12. The method of any one of claims 1-4, wherein the contacting is in vitro or in vivo.

13. The method of claim 3 or 4, wherein the subject suffers from alcoholism, diabetes or starvation.

14. The method of claim 3 or 4, wherein the subject suffers cirrhosis, steatosis or hepatocellular carcinoma.

15. A method for identifying an agent suitable for inhibiting replication of an RNA virus in a cell infected with the virus, wherein the cell is characterized as having been or concurrently being exposed to a physiologically relevant concentration of a compound selected from ethanol, acetate, isopropanol, acetaldehyde or acetone, comprising contacting a first sample of the cell with a candidate agent and separately contacting a second sample of the cell with contacting the cell with an effective amount of one or more of an HMG-CoA reductase inhibitor mevalonate pathway inhibitor, statins, a fatty acid biosynthesis inhibitor TOFA (5-(Tetradecyloxy)-2-furoic acid), cerulenin, thyroxine to decrease NADH/NAD+ ratio or an agent promoting clearance of the compound from a cell, wherein a decreased replication of the RNA virus in the cell substantially equal to or greater than the decreased replication of the RNA virsus in the second sample of the cell indicates that the candidate agent is suitable for inhibiting replication of the RNA virus in the cell.


SEQUENCE LISTING (3 sequences)

SEQ ID NO: 1 - DNA, 19 bp, Artificial Sequence
  Description of Artificial Sequence Synthetic primer
tctgcggaaccggtgagta

SEQ ID NO: 2 - DNA, 19 bp, Artificial Sequence
  Description of Artificial Sequence Synthetic primer
tcaggcagtaccacaaggc

SEQ ID NO: 3 - DNA, 21 bp, Artificial Sequence
  Description of Artificial Sequence Synthetic probe
ccagtcttcccggcaattccg